(19) United States
(12) Patent Application Publication
(10) Pub. No.: US 2024/0398779 A1
(43) Pub. Date: Dec. 5, 2024

(54) USE OF FACTOR B INHIBITORS FOR THE TREATMENT OF AGE-RELATED MACULAR DEGENERATION

(71) Applicant: Novartis AG, Basel (CH)

(72) Inventors: Arnaud Armel Olivier CHARIL, Swampscott, MA (US); Oliver SANDER, Basel (CH); Nadia ZAKARIA, Somerville, MA (US)

(21) Appl. No.: 18/689,933

(22) PCT Filed: Sep. 2, 2022
(86) PCT No.: PCT/IB2022/058275

§ 371 Date: Mar. 7, 2024

Related U.S. Application Data
(60) Provisional application No. 63/241,169, filed on Sep. 7, 2021.

(51) Int. Cl. A61K 31/454; A61K 9/00; A61P 27/02

(52) U.S. Cl. CPC A61K 31/454; A61K 9/0053; A61P 27/02

(57) ABSTRACT

Described herein are methods of treating early or intermediate age-related macular degeneration with the Factor B inhibitor iptacopan or a pharmaceutically acceptable salt thereof. e.g. iptacopan hydrochloride.

### FIELD

The disclosure relates to methods of treating complement driven diseases, and in particular, age-related macular degeneration with the Factor B inhibitor iptacopan or a pharmaceutically acceptable salt thereof, e.g. iptacopan hydrochloride.

### BACKGROUND

Age-related Macular Degeneration (AMD) is the leading cause of visual disability in the elderly population of the industrialized world with approximately 11 million people across the US affected with AMD with a global prevalence of 170 million (Pennington and DeAngelis Eye Vis (Lond), 34, 2016) and is expected to increase due to an ageing population.

The hallmark of early and intermediate AMD (c/iAMD) is the formation of drusen (lipid deposits containing complement proteins) and pigmentary changes in the macula, associated with mild to moderate loss of visual function, particularly under low luminance conditions (Schneck et al, Optom Vis Sci: 64-72, 2021). Early AMD is defined by drusen size ≥63 μm and ≤125 μm; and intermediate AMD by drusen >125 microns and/or pigmentary changes. High risk intermediate AMD patients have a 50% 5-year conversion risk to the late form of the disease: neovascular (n) AMD or geographic atrophy (GA) (Ferris et al, Arch Ophthalmol: 1570-4, 2005). Patients with GA have marked visual disability and continued progression to worsening vision and legal blindness (Chakravarthy et al, Ophthalmology: 842-849, 2018). Trials to slow GA have had limited effect, highlighting the need to treat earlier in the disease process (Guymer, Ophthalmol Retina: 515-517, 2018). Anti-VEGF injections can reduce vision loss from retinal fluid accumulation observed in nAMD, but do not prevent the underlying disease progression and development of atrophy. Although anti-VEGF reduces visual morbidity from nAMD, 98% of patients treated with IVT anti-VEGF therapy will go on to develop atrophy by 7 years follow-up (Rofagha et al, Ophthalmology: 2292-9, 2013). The lack of treatment for GA and the progression of nAMD to GA highlights the need to treat earlier in the disease.

Spectral Domain Optical Coherence Tomography (SD-OCT) has enabled the identification of high risk features that are highly predictive of progression to late AMD. These include intraretinal hyperreflective foci (which are thought to represent migration of retinal pigment epithelial cells (RPE) into the retina), hyporeflective foci within drusen (corresponding to calcific nodules), subretinal drusenoid deposits, and high central drusen volume. Recently, researchers (Lei et al, Arch Clin Exp Ophthalmol: 1551-1558, 2017) proposed a system using OCT images for integrating these factors into a simple score that could reflect a given patient's risk for conversion to late AMD. This system was later validated by Nassisi et al (Ophthalmology: 1667-1674 2019) in a post hoc analysis of non-late AMD fellow eyes from subjects enrolled in the HARBOR study. The non-late fellow eyes (including intermediate, early, or normal ageing) at baseline that had one or more high risk OCT features, displayed a two year conversion rate to advanced AMD between 33.1% to 84.4%.

Inflammatory and immune-mediated events involving complement proteins have been implicated in the biogenesis of drusen. Overactivation of the alternative complement pathway is strongly implicated in AMD pathology (Fritsche et al, Nat Genet: 433-9, 439c1-2, 2013). It is believed that the RPE-choroid as the key site of complement dysregulation in AMD. Inhibiting the alternative pathway (AP) in the RPE-choroid may slow or halt the loss of choroid dropout thereby enhancing blood flow to the RPE, enabling better nutrient transfer to the retina as well as improved clearance of retina/RPE waste products. Furthermore, inhibition of AP activation within drusen and at the Bruch's Membrane/RPE interface may improve the health and function of RPE cells and associated photoreceptors, thereby reducing disease activity in nAMD (Kauppien et al, Cell Mol Life Sci: 1765-86, 2016). An oral factor B inhibitor may reduce complement activation in the RPE-choroid, and may prevent progression of e/iAMD to atrophy and reduce nAMD disease activity.

LNP023, also known as iptacopan, is a novel, orally administered, small molecular weight, first-in-class, selective protease inhibitor that binds to Factor B (FB) Bb domain. FB is a key protease of the alternative pathway (AP) and the Bb domain is the active moiety of the AP C3 and C5 convertases. Inhibition of FB with oral iptacopan or a pharmaceutically acceptable salt thereof, e.g., iptacopan hydrochloride, has the potential to prevent or reduce formation of drusen and therefore reduce the conversion of e/iAMD, offering therapeutic benefits over and above the current standard of care (SoC). Additionally, the oral route of administration offers patients an advantage compared to the intraocular route of administration of current SoCs.

### SUMMARY

The disclosure relates to methods of treating complement driven diseases, and in particular, early or intermediate age-related macular degeneration with iptacopan or a pharmaceutically acceptable salt thereof, e.g. iptacopan hydrochloride. Iptacopan belongs to the class of Factor B inhibitors of the complement pathway and acts by inhibiting or suppressing the amplification of the complement system caused by C3 activation irrespective of the initial mechanism of activation. iptacopan hydrochloride is currently in clinical development for the treatment of paroxysmal nocturnal hemoglobinuria (PNH). Iptacopan hydrochloride is chemically designated as 4-((2S,4S)-(4-ethoxy-1-((5-methoxy-7-methyl-1H-indol-4-yl)methyl) piperidin-2-yl))benzoic acid hydrochloride and can be represented by the following chemical structure:

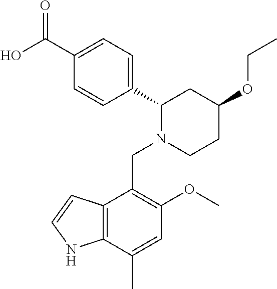 [SMILES: CCO[C@H]1CCN(Cc2c(OC)cc(C)c3[nH]ccc23)[C@H](c2ccc(C(=O)O)cc2)C1]

Iptacopan hydrochloride and methods for its preparation are disclosed in WO2015/009616 (see Example 26d), which is incorporated herein by reference in its entirety.

In one aspect, the disclosure provides a method of treating age-related macular degeneration (AMD) in an eye of a subject in need thereof, the method comprising orally administering to the subject, iptacopan or a pharmaceutically acceptable salt thereof, at a total dose of about 400 mg daily (wherein the dosing amount refers to the anhydrous free base of iptacopan). In some embodiments, the administration treats the subject, e.g., patient.

In another aspect, the disclosure provides a method of reducing incidence of progression of early or intermediate age-related macular degeneration (AMD) in a patient in need thereof, the method comprising orally administering to the subject, iptacopan or a pharmaceutically acceptable salt thereof, at a dose of about 400 mg daily (wherein the dosing amount refers to the anhydrous free base of iptacopan).

In another aspect, the disclosure provides a method of preventing progression of early or intermediate age-related macular degeneration (AMD) in a patient in need thereof, the method comprising orally administering to the subject, iptacopan or a pharmaceutically acceptable salt thereof, at a dose of about 400 mg daily (wherein the dosing amount refers to the anhydrous free base of iptacopan).

In another aspect, the disclosure provides a method of reducing incidence of atrophic lesions in a patient in need thereof, the method comprising orally administering to the subject, iptacopan or a pharmaceutically acceptable salt thereof, at a dose of about 400 mg daily (wherein the dosing amount refers to the anhydrous free base of iptacopan).

Treatment methods described herein can additionally comprise various evaluation steps prior to and/or following treatment with iptacopan or a pharmaceutically acceptable salt thereof, e.g. iptacopan hydrochloride. In an embodiment, prior to and/or after administration of iptacopan or a pharmaceutically acceptable salt thereof, e.g. iptacopan hydrochloride, the methods further comprise the step of evaluating PK and PD parameters (e.g., plasma concentration of iptacopan or a pharmaceutically acceptable salt thereof, e.g. iptacopan hydrochloride, C3, Fragment Bb, or sC5B). Evaluation may be achieved by sample analysis of bodily fluid, such as blood or plasma by e.g., mass spectroscopy, e.g. LC-MS.

### BRIEF DESCRIPTION OF THE DRAWINGS

FIG. 1 depicts a schematic of the study design.

FIG. 2 is a table of mobile MCVM sub study design.

### DETAILED DESCRIPTION

Described herein is the Phase 2 clinical study to determine safety and efficacy of iptacopan or a pharmaceutically acceptable salt thereof, e.g., iptacopan hydrochloride, in patients with early and intermediate age-related macular degeneration (e/iAMD). Accordingly, described herein are methods of treating e/iAMD in a patient in need thereof, the method comprising orally administering, e.g., in tablet or capsule form, to the patient a twice daily dose, e.g., about every 12 hours, of iptacopan or a pharmaceutically acceptable salt thereof, e.g., iptacopan hydrochloride, (wherein the dosing amount refers to the anhydrous free base of iptacopan hydrochloride). Also described herein are methods of selecting the target patient population, methods of monitoring treatment of the target patient population, and methods of assessing safety and efficacy of treatment of the target patient population.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings: As used herein, “about” means within +10% of a value.

As used herein, “administering” or “administration” means providing a pharmaceutical agent to an individual, and includes, but is not limited to, administering by a medical professional and self-administering. Administration of a pharmaceutical agent to an individual can be continuous, chronic, short or intermittent.

As used herein, the term “acquire” or “acquiring” as the terms are used herein, refer to obtaining possession of a physical entity (e.g., a sample, e.g., a blood sample or a blood plasma sample), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS, e.g., LC-MS/MS methods.

As used herein, “dose” means a specified quantity of a pharmaceutical agent provided in a single administration, or in a specified time period. In certain embodiments, a dose can be administered in capsules. As used herein, the dosing amount refers to the anhydrous free base of iptacopan hydrochloride.

As used herein, “individual”, “patient”, “participant”, or “subject” means a human selected for treatment or therapy.

As used herein, “pharmaceutically acceptable salts” means physiologically and pharmaceutically acceptable salts of iptacopan, i.e., salts that retain the desired biological activity of iptacopan and do not impart undesired toxicological effects thereto. The term “pharmaceutically acceptable salt” or “salt” includes a salt prepared from pharmaceutically acceptable non-toxic acids or bases, including inorganic or organic acids and bases.

“Pharmaceutically acceptable salts” of iptacopan may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). iptacopan hydrochloride and methods for its preparation are disclosed in WO2015/009616 (see Example 26d), which is incorporated herein by reference in its entirety.

As used herein, the term “treat” means decrease, suppress, attenuate, diminish, arrest, or stabilize the development or progression of a disorder or disease, e.g., e/iAMD.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### Methods of Use

In some embodiments, provided herein is a method of treating age-related macular degeneration (AMD) in an eye of a subject in need thereof, the method comprising orally administering to the subject, iptacopan or a pharmaceutically acceptable salt thereof, at a total dose of about 400 mg daily (wherein the dosing amount refers to the anhydrous free base of iptacopan). In some embodiments, the administration treats the subject, e.g., patient.

In one embodiment, the AMD is early AMD.

In one embodiment, the AMD is intermediate AMD.

In one embodiment, the iptacopan is administered twice a day.

In one embodiment, iptacopan is administered at a dose of 200 mg twice daily.

In one embodiment, iptacopan is administered at a dose of 100 mg four times daily.

In one embodiment, iptacopan is administered for at least one month.

In one embodiment, iptacopan is administered for at least six months.

In one embodiment, iptacopan is administered for at least one year.

In one embodiment, iptacopan is administered for about two years.

In one embodiment, the administration of iptacopan results in reduced vision loss in the eye compared to administration of placebo.

In one embodiment, the vision loss is measured by one or more of the following tests:
Early Treatment Diabetic Retinopathy Study (ETDRS) Best Corrected Visual Acuity (BCVA),
ETDRS Low Luminance Visual Acuity (LLVA),
Contrast Sensitivity (CS), or
Low Luminance Contrast Sensitivity (LLCS).

Best Corrected Visual Acuity (BCVA) may be measured using an ETDRS visual acuity chart at 4 meters or 1 meter for participants that cannot read the 4 meter chart. In some embodiments, the subject's ETDRS score for either the BCVA or LLVA assessment is greater by at least one line (5 letters), two lines (10 letters), or three lines (15 letters), compared to administration of a placebo.

In one embodiment, the administration of iptacopan reduces the formation of drusen in the eye compared to placebo. In some embodiments, the administration of iptacopan reduces the formation of drusen by about 10%, about 20%, about 30%, about 40%, or about 50%, compared to a placebo. In some embodiments, the administration of iptacopan reduces the formation of drusen by at least 10%, at least 20%, at least 30%, at least 40%, or at least 50%, compared to a placebo.

In one embodiment, the administration of iptacopan reduces the incidence of atrophic lesions in the eye of the subject. In some embodiments, the administration of iptacopan reduces the incidence of atrophic lesions by about 10%, about 20%, about 30%, about 40%, or about 50%, compared to a placebo. In some embodiments, the administration of iptacopan reduces the incidence of atrophic lesions by at least 10%, at least 20%, at least 30%, at least 40%, or at least 50%, compared to a placebo.

In one embodiment, the administration of iptacopan reduces the incidence and or size of Incomplete Retinal Pigment Epithelium and Outer Retinal Atrophy (iRORA) in the eye compared to the administration of placebo. In some embodiments, the administration of iptacopan reduces the incidence of iRORA by about 10%, about 20%, about 30%, about 40%, or about 50%, compared to a placebo. In some embodiments, the administration of iptacopan reduces the incidence of iRORA by at least 10%, at least 20%, at least 30%, at least 40%, or at least 50%, compared to a placebo.

In an embodiment, the subject, e.g., patient, has been diagnosed with early or intermediate AMD.

In an embodiment, the subject, e.g., patient, has been vaccinated prior to treatment with iptacopan or a pharmaceutically acceptable salt thereof, e.g., iptacopan hydrochloride.

In an embodiment, the subject, e.g., patient, has been vaccinated against Neisseria meningitidis (types A, C, Y and W-135) prior to treatment.

In some embodiments, provided herein is a method of reducing incidence of progression of early or intermediate age-related macular degeneration (AMD) in an eye of a patient in need thereof, the method comprising orally administering to the subject, iptacopan or a pharmaceutically acceptable salt thereof, at a dose of about 400 mg daily (wherein the dosing amount refers to the anhydrous free base of iptacopan).

In one embodiment, the iptacopan is administered twice a day.

In one embodiment, iptacopan is administered at a dose of 200 mg twice daily.

In one embodiment, iptacopan is administered at a dose of 100 mg four times daily.

In one embodiment, iptacopan is administered for at least one month.

In one embodiment, iptacopan is administered for at least six months.

In one embodiment, iptacopan is administered for at least one year.

In one embodiment, iptacopan is administered for about two years.

In one embodiment, the administration of iptacopan results in reduced vision loss in the eye compared to administration of placebo.

In one embodiment, the vision loss is measured by one or more of the following tests:
Early Treatment Diabetic Retinopathy Study (ETDRS) Best Corrected Visual Acuity (BCVA),
ETDRS Low Luminance Visual Acuity (LLVA),
Contrast Sensitivity (CS), or
Low Luminance Contrast Sensitivity (LLCS).

Best Corrected Visual Acuity (BCVA) may be measured using an ETDRS visual acuity chart at 4 meters or 1 meter for participants that cannot read the 4 meter chart. In some embodiments, the subject's ETDRS score for either the BCVA or LLVA assessment is greater by at least one line (5 letters), two lines (10 letters), or three lines (15 letters), compared to administration of a placebo.

In one embodiment, the administration of iptacopan reduces the formation of drusen in the eye, compared to placebo. In some embodiments, the administration of iptacopan reduces the formation of drusen by about 10%, about 20%, about 30%, about 40%, or about 50%, compared to a placebo. In some embodiments, the administration of iptacopan reduces the formation of drusen by at least 10%, at least 20%, at least 30%, at least 40%, or at least 50%, compared to a placebo.

In one embodiment, the administration of iptacopan reduces the incidence of atrophic lesions in the eye of the subject. In some embodiments, the administration of iptacopan reduces the incidence of atrophic lesions by about 10%, about 20%, about 30%, about 40%, or about 50%, compared to a placebo. In some embodiments, the administration of iptacopan reduces the incidence of atrophic lesions by at least 10%, at least 20%, at least 30%, at least 40%, or at least 50%, compared to a placebo.

In one embodiment, the administration of iptacopan reduces the incidence and or size of Incomplete Retinal Pigment Epithelium and Outer Retinal Atrophy (iRORA) in the eye compared to the administration of placebo. In some embodiments, the administration of iptacopan reduces the incidence of iRORA by about 10%, about 20%, about 30%, about 40%, or about 50%, compared to a placebo. In some embodiments, the administration of iptacopan reduces the incidence of iRORA by at least 10%, at least 20%, at least 30%, at least 40%, or at least 50%, compared to a placebo.

In an embodiment, the subject, e.g., patient, has been vaccinated prior to treatment with iptacopan or a pharmaceutically acceptable salt thereof, e.g., iptacopan hydrochloride.

In an embodiment, the subject, e.g., patient, has been vaccinated against Neisseria meningitidis (types A, C, Y and W-135) prior to treatment.

In some embodiments, provided herein is a method of preventing progression of early or intermediate age-related macular degeneration (AMD) in an eye of a patient in need thereof, the method comprising orally administering to the subject, iptacopan or a pharmaceutically acceptable salt thereof, at a dose of about 400 mg daily (wherein the dosing amount refers to the anhydrous free base of iptacopan).

In one embodiment, the iptacopan is administered twice a day.

In one embodiment, iptacopan is administered at a dose of 200 mg twice daily.

In one embodiment, iptacopan is administered at a dose of 100 mg four times daily.

In one embodiment, iptacopan is administered for at least one month.

In one embodiment, iptacopan is administered for at least six months.

In one embodiment, iptacopan is administered for at least one year.

In one embodiment, iptacopan is administered for about two years.

In one embodiment, the administration of iptacopan results in reduced vision loss in the eye compared to administration of placebo.

In one embodiment, the vision loss is measured by one or more of the following tests:
Early Treatment Diabetic Retinopathy Study (ETDRS) Best Corrected Visual Acuity (BCVA),
ETDRS Low Luminance Visual Acuity (LLVA),
Contrast Sensitivity (CS), or
Low Luminance Contrast Sensitivity (LLCS).

Best Corrected Visual Acuity (BCVA) may be measured using an ETDRS visual acuity chart at 4 meters or 1 meter for participants that cannot read the 4 meter chart. In some embodiments, the subject's ETDRS score for either the BCVA or LLVA assessment is greater by at least one line (5 letters), two lines (10 letters), or three lines (15 letters), compared to administration of a placebo.

In one embodiment, the administration of iptacopan reduces the formation of drusen in the eye, compared to placebo. In some embodiments, the administration of iptacopan reduces the formation of drusen by about 10%, about 20%, about 30%, about 40%, or about 50%, compared to a placebo. In some embodiments, the administration of iptacopan reduces the formation of drusen by at least 10%, at least 20%, at least 30%, at least 40%, or at least 50%, compared to a placebo.

In one embodiment, the administration of iptacopan reduces the incidence of atrophic lesions in the eye of the subject. In some embodiments, the administration of iptacopan reduces the incidence of atrophic lesions by about 10%, about 20%, about 30%, about 40%, or about 50%, compared to a placebo. In some embodiments, the administration of iptacopan reduces the incidence of atrophic lesions by at least 10%, at least 20%, at least 30%, at least 40%, or at least 50%, compared to a placebo.

In one embodiment, the administration of iptacopan reduces the incidence and or size of Incomplete Retinal Pigment Epithelium and Outer Retinal Atrophy (iRORA) in the eye compared to the administration of placebo. In some embodiments, the administration of iptacopan reduces the incidence of iRORA by about 10%, about 20%, about 30%, about 40%, or about 50%, compared to a placebo. In some embodiments, the administration of iptacopan reduces the incidence of iRORA by at least 10%, at least 20%, at least 30%, at least 40%, or at least 50%, compared to a placebo.

In an embodiment, the subject, e.g., patient, has been vaccinated prior to treatment with iptacopan or a pharmaceutically acceptable salt thereof, e.g., iptacopan hydrochloride.

In some embodiments, provided herein is a method of reducing incidence of atrophic lesions in an eye of a patient in need thereof, the method comprising orally administering to the subject, iptacopan or a pharmaceutically acceptable salt thereof, at a dose of about 400 mg daily (wherein the dosing amount refers to the anhydrous free base of iptacopan). In some embodiments, provided herein is a method of preventing atrophic lesions in an eye of a patient in need thereof, the method comprising orally administering to the subject, iptacopan or a pharmaceutically acceptable salt thereof, at a dose of about 400 mg daily (wherein the dosing amount refers to the anhydrous free base of iptacopan).

In one embodiment, the iptacopan is administered twice a day.

In one embodiment, iptacopan is administered at a dose of 200 mg twice daily.

In one embodiment, iptacopan is administered at a dose of 100 mg four times daily.

In one embodiment, iptacopan is administered for at least one month.

In one embodiment, iptacopan is administered for at least six months.

In one embodiment, iptacopan is administered for at least one year.

In one embodiment, iptacopan is administered for about two years.

In one embodiment, the administration of iptacopan results in reduced vision loss in the eye compared to administration of placebo.

In one embodiment, the vision loss is measured by one or more of the following tests:
Early Treatment Diabetic Retinopathy Study (ETDRS) Best Corrected Visual Acuity (BCVA),
ETDRS Low Luminance Visual Acuity (LLVA),
Contrast Sensitivity (CS), or
Low Luminance Contrast Sensitivity (LLCS).

Best Corrected Visual Acuity (BCVA) may be measured using an ETDRS visual acuity chart at 4 meters or 1 meter for participants that cannot read the 4 meter chart. In some embodiments, the subject's ETDRS score for either the BCVA or LLVA assessment is greater by at least one line (5 letters), two lines (10 letters), or three lines (15 letters), compared to administration of a placebo.

In one embodiment, the administration of iptacopan reduces the formation of drusen in the eye compared to placebo. In some embodiments, the administration of iptacopan reduces the formation of drusen by about 10%, about 20%, about 30%, about 40%, or about 50%, compared to a placebo. In some embodiments, the administration of iptacopan reduces the formation of drusen by at least 10%, at least 20%, at least 30%, at least 40%, or at least 50%, compared to a placebo.

In some embodiments, the administration of iptacopan reduces the incidence of atrophic lesions in the eye by about 10%, about 20%, about 30%, about 40%, or about 50%, compared to a placebo. In some embodiments, the administration of iptacopan reduces the incidence of atrophic lesions by at least 10%, at least 20%, at least 30%, at least 40%, or at least 50%, compared to a placebo.

In one embodiment, the administration of iptacopan reduces the incidence and or size of Incomplete Retinal Pigment Epithelium and Outer Retinal Atrophy (iRORA) in the eye compared to the administration of placebo. In some embodiments, the administration of iptacopan reduces the incidence of iRORA by about 10%, about 20%, about 30%, about 40%, or about 50%, compared to a placebo. In some embodiments, the administration of iptacopan reduces the incidence of iRORA by at least 10%, at least 20%, at least 30%, at least 40%, or at least 50%, compared to a placebo.

In an embodiment, the subject, e.g., patient, has been vaccinated prior to treatment with iptacopan or a pharmaceutically acceptable salt thereof, e.g., iptacopan hydrochloride.

In any of the embodiments described herein, the subject may be vaccinated prior to administration of iptacopan or a pharmaceutically acceptable salt thereof against Neisseria meningitidis (types A, C, Y and W-135)

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### LIST OF ABBREVIATIONS

AE Adverse Event
AESI Adverse Events of Special Interest
ALP Alkaline Phosphatase
ALT Alanine Aminotransferase
AMD Age-Related Macular Degeneration
AP Alternate Pathway
AST Aspartate Aminotransferase
b.i.d. bis in die/twice a day
BCVA Best Corrected Visual Acuity
BMI Body Mass Index
BUN Blood Urea Nitrogen
C3G Complement 3 Glomerulopathy
CC Choriocapillaries
CFH Complement Factor H
CFP Color Fundus Photography
CK Creatinine Kinase
CMO & PS Chief Medical Office and Patient Safety
CO Country Organization
CP Classical Pathway
CRF Case Report/Record Form (paper or electronic)
CRO Contract Research Organization
CRORA Complete Retinal Pigment Epithelium and Outer Retinal Atrophy
CS Contrast Sensitivity
CSR Clinical Study Report
DIN Drug Induced Nephrotoxicity
DMC Data Monitoring Committee
DNA Deoxyribonucleic Acid
eAMD Early AMD
ECG Electrocardiogram
EDC Electronic Data Capture
ELISA Enzyme-linked Immunosorbent Assay
eSource Electronic Source
ETDRS Early Treatment Diabetic Retinopathy Study
FA Fluorescein Angiography
FAF Fundus Autofluorescence
FB Factor B
FDA Food and Drug Administration
FIH First in Human
FSH Follicle Stimulating Hormone
GA Geographic Atrophy
GCP Good Clinical Practice
GCS Global Clinical Supply
GGT Gamma-glutamyl transferase
GLP Good Laboratory Practices
h Hour
HBcAb Hepatitis B Core Antibody
HBsAg Hepatitis B Surface Antigen
HBV Hepatitis B Virus
HCV Hepatitis C Virus
HIV Human Immunodeficiency Virus
iAMD Intermediate AMD
IB Investigator's Brochure
ICF Informed Consent Form
ICH International Council for Harmonization of Technical Requirements for
Pharmaceuticals for Human Use
IEC Independent Ethics Committee
INR International Normalized Ratio
IOP Intraocular Pressure
IR Infrared
IRB Institutional Review Board
iRORA Incomplete Retinal Pigment Epithelium and Outer Retinal Atrophy
IRT Interactive Response Technology
IVT Intravitreal
LDH Lactate Dehydrogenase
LFT Liver function test
LLCS Low Luminance Contrast Sensitivity
LLN Lower Limit of Normal
LLOQ Lower Limit of Quantification
LOAEL Lowest Observed Adverse Effect Level
MAC Membrane Attack Complex
MCVM Manifold Contrast Vision Meter
MedDRA Medical Dictionary for Regulatory Activities
mg Milligram(s)
mL Milliliter(s)
MNV Macular Neovascularization
mRNA Messenger-Ribonucleic Acid
MTD Maximum Tolerated Dose
nAMD Neovascular AMD
OCT Optical Coherence Tomography
OCT A Optical Coherence Tomography Angiography
OHP Off-site Healthcare Professional
Participant Participant Number
No
PD Pharmacodynamic(s)
PK Pharmacokinetic(s)
PNH Paroxysmal Nocturnal Hemoglobinuria
PRO Patient Reported Outcomes
qCSF Quantitative Contrast Sensitivity Function
QMS Quality Management System
QTcF QT interval corrected by Fridericia's formula
R Value ALT/ALP×ULN
RBC Red Blood Cell(s)
RPE Retinal Pigment Epithelium
SAE Serious Adverse Event
SAP Statistical Analysis Plan
sCR Serum Creatinine
SD Standard Deviation
SD-OCT Spectral Domain Optical Coherence Tomography
SGOT Serum Glutamic Oxaloacetic Transaminase
SGPT Serum Glutamic Pyruvic Transaminase
SNP Single Nucleotide Polymorphism
SS-OCT Swept Source Optical Coherence Tomography
SUSAR Suspected Unexpected Serious Adverse Reaction
TB Tuberculosis
ULN Upper Limit of Normal
USV Unscheduled Visit
VEGF Vascular Endothelial Growth Factor
VFQ Visual Function Questionnaire
VILL Vision Impairment in Low Luminance
WBC White Blood Cell(s)
WHO World Health Organization
WoC Withdrawal of Consent

Glossary of terms

| | |
|---|---|
| Additional | Medicinal products that may be used during the clinical trial as |
| treatment | described in the protocol, but not as an investigational medicinal product |
| | (e.g. any background therapy) |
| Assessment | A procedure used to generate data required by the study |
| Biologic Samples | A biological specimen including, for example, blood (plasma, serum), |
| | saliva, tissue, urine, stool, etc. taken from a study participant |
| Cohort | A specific group of participants fulfilling certain criteria and generally |
| | treated at the same time |
| Control drug | A study drug (active or placebo) used as a comparator to reduce |
| | assessment bias, preserve blinding of investigational drug, assess |
| | internal study validity, and/or evaluate comparative effects of the |
| | investigational drug |
| Dosage | Dose of the study treatment given to the participant in a time unit (e.g. |
| | 100 mg once a day, 75 mg twice a day) |
| Electronic Data | Electronic data capture (EDC) is the electronic acquisition of clinical |
| Capture (EDC) | study data using data collection systems, such as Web-based |
| | applications, interactive voice response systems and clinical laboratory |
| | interfaces. EDC includes the use of Electronic Case Report Forms |
| | (eCRFs) which are used to capture data transcribed from paper source |
| | forms used at the point of care |
| End of the clinical | The end of the clinical trial is defined as the last visit of the last |
| trial | participant or at a later point in time as defined by the protocol |
| Enrollment | Point/time of participant entry into the study at which informed consent |
| | must be obtained |
| Estimand | A precise description of the treatment effect reflecting the clinical |
| | question posed by the trial objective. It summarizes at a population-level |
| | what the outcomes would be in the same patients under different |
| | treatment conditions being compared. Attributes of an estimand include |
| | the population, variable (or endpoint) and treatment of interest, as well |
| | as the specification of how the remaining intercurrent events are |
| | addressed and a population-level summary for the variable. |
| Healthy volunteer | A person with no known significant health problems who volunteers to |
| | be a study participant |
| Intercurrent events | Events occurring after treatment initiation that affect either the |
| | interpretation or the existence of the measurements associated with the |
| | clinical question of interest. |
| Investigational | The drug whose properties are being tested in the study |
| drug/treatment | |
| Medication | A unique identifier on the label of medication kits |
| number | |
| Mis-randomized | Mis-randomized participants are those who were not qualified for |
| participants | randomization and who did not take study treatment, but have been |
| | inadvertently randomized into the study |
| Other treatment | Treatment that may be needed/allowed during the conduct of the study |
| | (i.e. concomitant or rescue therapy) |
| Part | A sub-division of a study used to evaluate specific objectives or contain |
| | different populations. For example, one study could contain a single |
| | dose part and a multiple dose part, or a part in participants with |
| | established disease and in those with newly-diagnosed disease |
| Participant | A trial participant (can be a healthy volunteer or a patient) |
| Participant number | A unique number assigned to each participant upon signing the informed |
| | consent. This number is the definitive, unique identifier for the |
| | participant and should be used to identify the participant throughout the |
| | study for all data collected, sample labels, etc. |
| Period | The subdivisions of the trial design (e.g. Screening, Treatment, Follow- |
| | up) which are described in the Protocol. Periods define the study phases |
| | and will be used in clinical trial database setup and eventually in |
| | analysis |
| Personal data | Participant information collected by the Investigator that is coded and |
| | transferred to Novartis for the purpose of the clinical trial. This data |
| | includes participant identifier information, study information and |
| | biological samples. |
| Premature | Point/time when the participant exits from the study prior to the planned |
| participant | completion of all study drug administration and/or assessments; at this |
| withdrawal | time all study drug administration is discontinued and no further |
| | assessments are planned |
| Randomization | A unique identifier assigned to each randomized participant |
| number | |
| Screen Failure | A participant who did not meet one or more criteria that were required |
| | for participation in the study |
| Source | Source data refers to the initial record, document, or primary location |
| Data/Document | from where data comes. The data source can be a database, a dataset, a |
| | spreadsheet or even hard-coded data, such as paper or eSource |
| Start of the clinical | The start of the clinical trial is defined as the signature of the informed |
| trial | consent by the first participant |
| Study treatment | Any drug or combination of drugs or intervention administered to the |
| | study participants as part of the required study procedures; includes |
| | investigational drug(s), control(s) or background therapy |
| Study treatment | When the participant permanently stops taking any of the study drug(s) |
| discontinuation | prior to the defined study treatment completion date (if any) for any |
| | reason; may or may not also be the point/time of study discontinuation |
| Treatment | A treatment arm/group defines the dose and regimen or the combination, |
| arm/group | and may consist of 1 or more cohorts. |
| Treatment of | The treatment of interest and, as appropriate, the alternative treatment to |
| interest | which comparison will be made. These might be individual |
| | interventions, combinations of interventions administered concurrently, |
| | e.g. as add-on to standard of care, or might consist of an overall regimen |
| | involving a complex sequence of interventions. This is the treatment of |
| | interest used in describing the related clinical question of interest, which |
| | might or might not be the same as the study treatment. |
| Variable (or | The variable (or endpoint) to be obtained for each participant that is |
| endpoint) | required to address the clinical question. The specification of the |
| | variable might include whether the participant experiences an |
| | intercurrent event. |
| Withdrawal of | Withdrawal of consent from the study occurs only when a participant |
| study consent | does not want to participate in the study any longer and does not allow |
| (WoC) | any further collection of personal data |

Example 1. A Randomized, Participant and Investigator Masked, Placebo-Controlled, Multicenter, Proof-of-Concept Study to Assess the Safety and Efficacy of Iptacopan in Patients with Early and Intermediate Age-Related Macular Degeneration

### Purpose

The purpose of this study is to assess the effect of iptacopan to prevent conversion of early or intermediate AMD eyes to new incomplete retinal pigment epithelium and outer retinal atrophy (iRORA) or late AMD.

Objectives and Endpoints

| Objective(s) | Endpoint(s) |
|---|---|
| Primary objective(s) | Endpoint(s) for primary objective(s) |
| To evaluate the effect of iptacopan to | Development of new iRORA or late |
| prevent conversion of early or intermediate | AMD, (either geographic atrophy or |
| AMD eyes to new iRORA or late AMD | neovascular AMD) in the e/iAMD eye |
| | from Baseline/Day 1 through Month 24, |
| | as determined by OCT and supported by |
| | multimodal imaging |
| Secondary objective(s) | Endpoint(s) for secondary objective(s) |
| To assess the safety and tolerability of | Incidence of ocular and non-ocular AEs |
| iptacopan in the study population | (including systemic safety measures) |
| | from Baseline/Day 1 through Month 24 |
| To assess the effect of iptacopan on visual | Change in visual function from |
| function in the e/iAMD eye | Baseline/Day 1 through Month 24 as |
| | measured by: |
| | ETDRS Best Corrected Visual |
| | Acuity (BCVA) |
| | ETDRS Low Luminance Visual |
| | Acuity (LLVA) |
| | Contrast Sensitivity (CS) |
| | Low Luminance Contrast Sensitivity |
| | (LLCS) |
| To assess iptacopan pharmacokinetics | Parameters related to total parent drug, |
| | including but not limited to Tmax, Cmax |
| | and AUC. |
| Exploratory objective(s) | Endpoint(s) for exploratory objective(s) |
| To assess additional vascular pathology | Change in choriocapillaris flow voids |
| changes in both eyes | from Baseline/Day 1 to each post |
| | Baseline visit |
| | Incidence of non-exudative subclinical |
| | MNV from Baseline/Day 1 to each post |
| | Baseline visit |
| To assess the frequency of IVT injections | Frequency of Standard of Care |
| in the nAMD eye | IVT injections from Baseline/Day 1 to |
| | Month 24 |
| To explore contrast sensitivity changes | Changes of contrast sensitivity (high and |
| using the stationary Manifold Contrast | low luminance) at specified spatial |
| Vision Meter (MCVM) in both eyes | frequencies from Baseline/Day to each |
| | post Baseline visit. |
| To explore the use of the mobile MCVM in | Ability of participants to use the mobile |
| assessing contrast sensitivity at home in the | MCVM at home (quality testing and |
| study eye and in the clinic in both eyes | compliance to the instructions). |
| from Baseline/Day 1 to Month 2 (at | Agreement of contrast sensitivity at |
| selected sites) | specified spatial frequencies obtained |
| | with the mobile MCVM in the clinic and |
| | stationary MCVM in the clinic at |
| | Baseline/Day 1 and Month 2 visits. |
| To assess the effect of iptacopan on visual | Change in visual function in the nAMD |
| function in the nAMD eye | eye from Baseline/Day 1 through Month |
| | 24 as measured by: |
| | ETDRS Best Corrected Visual |
| | Acuity |
| To assess the effect of iptacopan on the | ETDRS Low Luminance Visual |
| incidence of atrophic lesions in the nAMD | Acuity |
| eye | Contrast Sensitivity |
| | Low Luminance Contrast Sensitivity |
| To assess the effect of iptacopan | Incidence and change in size of iRORA |
| on atrophic lesion growth in the nAMD | as determined by multimodal imaging |
| eye | Incidence and change in size of complete |
| | retinal pigment epithelium and outer |
| | retinal atrophy (cRORA) as determined |
| | by multimodal imaging |
| To assess the effect of iptacopan on patient | Scores of the National Eye Institute |
| reported outcomes | Visual Function Questionnaire-25 (NEI- |
| | VFQ-25) from Baseline/Day 1 through |
| | Month 24 |
| | Scores of the VILL (Visual Impairment |
| | in Low Luminance) Questionnaire from |
| | Baseline/Day 1 through Month 24 |
| To explore potential genetic factors that | Due to the exploratory nature of this |
| may influence e/iAMD conversion to new | objective additional endpoints are not |
| iRORA or late AMD | predefined |
| To assess longitudinal changes of disease | Complement pathway biomarkers such |
| and pathway biomarkers and their | as but not limited to: |
| relationship to clinical outcome. | Wieslab ® assay, |
| | Complement factor Bb, Factor B, |
| | and C4d in plasma |
| To assess the time until conversion to new | if feasible, Factor B, sC5b9, Bb |
| iRORA or late AMD | and/or C4d in aqueous humor |
| | Time to conversion from e/iAMD to new |
| | iRORA or late AMD (as defined for the |
| | primary endpoint) from Baseline/Day 1 |
| | through Month 24 |

### Study Design

This is a 29-month, multicenter, randomized, participant and investigator masked, placebo controlled, proof-of-concept study to assess the safety and efficacy of iptacopan in participants with early to intermediate age-related macular degeneration (e/iAMD) in one eye and neovascular age-related macular degeneration (nAMD) in the other eye. All enrolled participants must have e/iAMD in one eye, with at least one high risk OCT feature (study eye) and nAMD in the other eye (fellow eye).

Participants who consent will undergo screening assessments to evaluate their eligibility based on the inclusion and exclusion criteria. Participants who meet all of the eligibility criteria will be randomized at the Baseline/Day 1 visit in a 1:1 ratio into one of two treatment arms:
Iptacopan 200 mg, b.i.d, oral
Placebo, b.i.d, oral

Participants will be stratified between the two arms for the presence of hyper-reflective foci as confirmed on optical coherence tomography (OCT) by the central reading center.

For both arms of the study, the fellow eye (nAMD eye) will be treated with anti VEGF therapy according to current local standard of care.

Approximately 146 participants (73 per arm) will be treated worldwide. The maximum study duration for one participant is approximately 880 days, including screening and post treatment follow up.

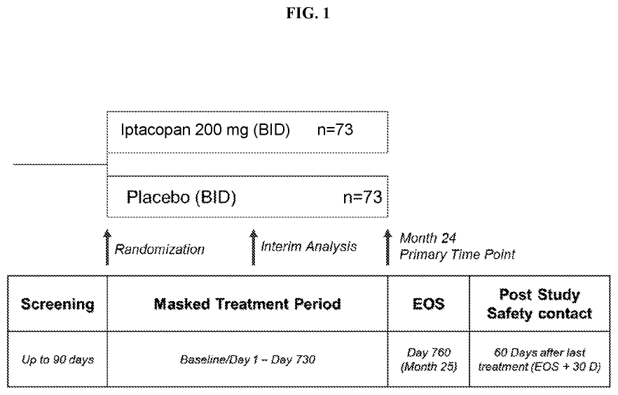

There are 3 periods in this study (see FIG. 1):
Screening period: Day-90 to Day-1.
Treatment period: Baseline/Day 1 through Day 730.
Follow Up period: (EOS): Day 760/Month 25 and Post study safety contact 30 days after the EOS visit (approximately 60 days after last treatment)

### Screening Period: Day −90 to Day −1

A screening period of up to 90 days will be used to assess eligibility.

At the screening visit, following the administration of the informed consent forms, assessments will be taken to determine eligibility, including demography, medical history, vital signs, height and weight, a physical exam, an ECG, an assessment of pregnancy, multiple imaging and vision tests and blood samples to assess the participants general health and adherence to inclusion/exclusion criteria. During the screening period, biological samples and imaging assessments will be evaluated to assess eligibility.

Once all eligibility requirements have been met and if the participant has not been vaccinated (or requires booster) against N. meningitidis. S. pneumoniae and H. influenzae then arrangements for these vaccinations will be made at least 2 weeks prior to Baseline/Day 1 visit. The logistics of vaccination administration will be managed by the investigating site. The sponsor may provide support if needed.

Rescreening of participants is allowed. Imaging assessments may be redone to enhance quality if difficult to interpret, as advised by the central reading center.

The retesting of an assessment(s) may be repeated within the screening window, within up to 89 days of the initial screening visit. Other screening assessments that passed initially do not need to be repeated. If rescreening occurs outside of the screening window, greater than 89 days from the original screening visit date, then all screening procedures must be repeated.

### Investigator/Participant Masked Treatment Period: Baseline/Day 1 Through Month 24/Day 730

On the Baseline/Day1 visit, eligible participants will be randomized to one of the treatment arms: treatment of iptacopan or placebo in a 1/1 ratio. A study visit schedule will be established at the time of randomization for all participants. Except for the Baseline/Day 1 visit, assessments may be performed on two separate days, provided both days are in the visit window and within 3 days of each other. At Baseline/Day 1, the first dose of the study treatment will be administered on site before the participant leaves the clinic. Following the first dose, participants will self administer study treatment (morning and evening) during the 24 months of the treatment period and will be seen at 11 follow-up visits.

On clinic visit days, dosing should occur AFTER the participant has completed the questionnaires, has had vital signs, ECGs and all blood samples taken. It is not necessary to complete the ocular examination, imaging assessments, ETDRS visual acuity and contrast sensitivity measurements prior to dosing.

At the treatment visits, some or all of the following assessments will be taken to evaluate safety and efficacy of the treatment: vital signs, height and weight, physical exams, ECGs, pregnancy tests, multiple imaging and vision tests including questionnaires, and blood samples to assess the participant's general health and adherence to the treatment. In addition, participants will be queried about their general health and the occurrence of any adverse events. At all study visits during the treatment period, participants will return their study medication, including bottles, any unused medication, and the participant diary for use by the site to perform drug accountability. Participants will be resupplied with study medication at specified visits.

Final self administration of study drug will occur the night before the Month 24 (Day 30) visit (end of treatment).

### End of Study (EOS) Visit: Month 25/Day 760

All participants will be seen on Month 25/Day 760 for the EOS visit. At this visit, all end of study assessments will be completed, including the study completion information.

### Follow-Up Period: (Post Study Safety Contact) Month 26/Day 790

A post-treatment safety telephone call will be performed approximately 30 days after the EOS (Month 25/Day 760) visit.

### Mobile MCVM Testing

At select sites, participants who meet all the screening requirements and who will be eligible to move on to Baseline/Day 1 randomization, will be asked to join a separate 2-month sub-study designed to compare the mobile MCVM to the stationary MCVM device used in the clinic. The study population for mobile MCVM testing will consist of approximately 50 male and female participants who consent to this sub-study.

Examining assessments of bilateral contrast sensitivity will be tested using the mobile MCVM in the clinic at three visits, including Screening, Baseline/Day 1 and Month 2. Duplicate testing for reproducibility will occur at the Baseline/Day 1 and Month 2 visits.

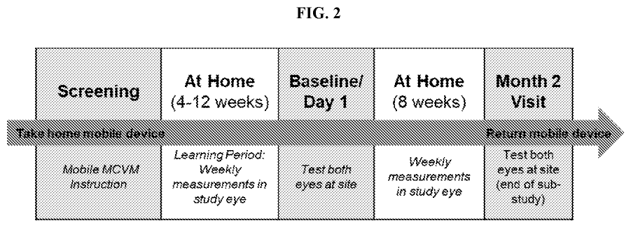

In addition to the assessments in the clinic, the participants will also use the mobile MCVM at home to perform two measurements in the study eye each week, taken 5-10 minutes apart in between the Screening to Month 2 visits (same time of day and same location are recommended). For the weeks when a clinic visit is scheduled, home testing with the mobile MCVM is recommended to be performed on the same day as the clinic visit. The sub-study workflow is represented in FIG. 2.

### Rationale for Study Design

The design of this study will address the objectives of efficacy and safety of oral, 200 mg b.i.d iptacopan for the treatment of e/iAMD.

The study will be participant and investigator masked and the Sponsor will remain unmasked (emergency unmasking procedures will be available in the event of an adverse event that requires knowledge of treatment arm).

The primary endpoint of conversion to new iRORA or late AMD is based on objective evaluations by the central reading facility which will also be masked to the treatment assignment. Intraretinal hyperreflective foci, as observed on OCT, are the most predictive risk factor for conversion, therefore the participants with this risk factor will be equally randomized between the iptacopan and placebo arm. The two-year study treatment period is based on publications on conversion to late AMD in patients with high risk factors (Lei et al, Graefes Arch Clin Exp Ophthalmol: 1551-1558, 2017, Nassisi et al, Graefes Arch Clin Exp Ophthalmol: 2079-2085, 2019).

Treatment randomization between the active and placebo arm allows the evaluation of safety and efficacy characteristics in an objective manner. All study participants will undergo the same study assessments and receive their assigned study treatments under the same dosing and visit intervals. Both treatment arms will proceed in parallel to each other.

### Rationale for Dose/Regimen and Duration of Treatment

Since e/iAMD is a slowly progressive disease, prolonged treatment and observation are required to detect potential clinical efficacy with iptacopan. A 24-month study was selected in order to allow adequate time to observe the time to conversion of the e/iAMD eyes treated with iptacopan as compared to e/iAMD eyes treated with placebo.

Iptacopan at 200 mg b.i.d. has been selected for this study based on the totality of safety and efficacy data from the healthy volunteer studies and a Phase 2 study Part 1 interim analysis. In addition, this dose has demonstrated a favorable benefit-risk ratio in phase 2 studies in C3G including proteinuria reduction as well as PNH patients to maintain hemoglobin (Hb) levels.

In first in human studies, iptacopan was administered to 102 healthy volunteers in single ascending dose (SAD, 10 to 400 mg) and multiple ascending dose (MAD, 10 to 200 mg b.i.d. for 2 weeks). The results showed that iptacopan had high solubility, good permeability, a fast absorption, and was well tolerated. No deaths, SAEs, or AEs leading to study drug discontinuation were observed. In the MAD study, dose dependent suppression of AP activity was achieved when measured by Wieslab AP assay, with approximately 80% or greater inhibition sustained over 14 days of dosing at 100 mg and 200 mg b.i.d, while Bb level showed 30-40% decrease from the baseline for all iptacopan cohorts.

Clinical phase 2 data showed that in all tested indications (PNH, IgAN, C3G), the 200 mg b.i.d. dose regimen provided the highest response rate without any safety concerns. This was also supported by PKPD modeling as well as target occupancy data collected in these studies. Consequently for this study, which serves to investigate efficacy and PK characterization in a new participant population, 200 mg b.i.d. was selected.

In addition, the preclinical studies supported acceptable safety margins for human exposure following 200 mg b.i.d. dosing.

The unbound systemic exposures (AUC-based) in participants at 200 mg b.i.d. will stay 17.3-fold below the lowest exposures at which off-target testicular effects were observed (LOAEL) in the 39-week dog study.

Safety margins based on unbound systemic exposures lead to a calculated ratio of 2.2 (AUC- and Cmax-based) for the most sensitive species and gender (NOAEL) in male dogs in the 39-week toxicity study.

The safety and tolerability of the iptacopan dose at 200 mg b.i.d. were supported by the Part 1 of study in IgAN, as well as the other clinical studies in PNH and C3G for which iptacopan 200 mg b.i.d. was administered for more than 12 months in some participants. In Part 1 of the study, iptacopan dose at 200 mg b.i.d. was shown to reduce proteinuria over 90 days supporting 200 mg b.i.d. of iptacopan.

### Rationale for Choice of Control Drugs (Comparator/Placebo) or Combination Drugs

Since there is no product approved for the treatment of e/iAMD, no active comparator is included in the current study. The control for this study will be placebo treatment, which is a common and well-established method of control for studies of oral therapies with no approved treatment.

### Study Population

The study population under investigation consists of male and female participants ≥50 years old diagnosed with e/iAMD in the study eye and nAMD in the fellow eye. The study eye in addition to having at least early AMD, must have at least one high risk feature on OCT (intraretinal hyperreflective foci, subretinal drusenoid deposits, hypo-reflective foci within drusen or drusen volume ≥0.03 mm³ within the central 3 mm circle). The fellow eye must have a history of nAMD and have been on (or will be by Baseline/Day 1) Standard of Care anti-VEGF intravitreal treatment. Approximately 292 participants will be screened and 146 participants randomized (n=73 in each study arm) in a 1:1 ratio worldwide.

The investigators ensure that all participants being considered for the study meet the eligibility criteria. No additional criterion is to be applied by the investigators, in order to ensure that the study population is representative of all eligible participants.

### Inclusion Criteria

Participants eligible for inclusion in this study must meet all of the following criteria prior to Baseline/Day 1:
1. Written informed consent must be obtained before any assessment is performed.
2. Male or female participants ≥50 years of age.
3. Diagnosis of early or intermediate AMD in the study eye as determined by the investigator on fundus examination (color fundus photographs will not require confirmatory over read by central reading center).
4. Study eye (e/iAMD eye) must have at least one of the following OCT features: intraretinal hyperreflective foci, subretinal drusenoid deposits, hypo-reflective foci within drusen or drusen volume ≥0.03 mm³ within the central 3 mm circle on OCT as determined by the central reading center.
5. Diagnosis of nAMD in the fellow eye as determined by the investigator:
   nAMD eye must have a history of exudative MNV (Type 1, 2 or 3) and treatment with anti-VEGF intravitreal injections. Newly diagnosed nAMD who receive their first anti-VEGF treatment at the Baseline/Day 1 visit are eligible.
6. Vaccination against Neisseria meningitidis and Streptococcus pneumoniae infection are required prior to the start of the treatment with iptacopan. If the participant has not been previously vaccinated, or if a booster is required, vaccines should be given according to local regulations, at least 2 weeks prior to first iptacopan dosing.
7. If not received previously, vaccination against Haemophilus influenzae infection should be given, if available and according to local regulations. The vaccine should be given at least 2 weeks prior to first iptacopan dosing.
8. Able to communicate well with the investigator, to understand and comply with the requirements of the study.

### Exclusion Criteria

Participants meeting any of the following criteria are not eligible for inclusion in this study.
1. Concomitant medical or ocular conditions, at Screening or Baseline/Day 1 which could, in the opinion of the Investigator, prevent response to study treatment, may confound interpretation of study results, compromise visual acuity, require planned medical or surgical intervention during the study period (e.g. cataract surgery), preclude scheduled study visits, completion of the study, or safe administration of the investigational product.
2. Intraocular surgery, including cataract and vitreoretinal surgery, in the study eye within 3 months prior to Baseline/Day 1.
3. Presence of significant media opacity, eye movement disorder (nystagmus), severe ptosis, extraocular motility restriction or head tremor, which in the opinion of the investigator, would prevent adequate fundus visualization or interfere with retinal imaging data quality.
4. Any active intraocular or periocular infection or active intraocular inflammation (e.g. infection conjuntivitis, keratitis, scleritis, endophthalmitis, infectious blepharitis, uveitis) in either eye at Screening or Baseline/Day 1.
5. Presence or suspicion (based on the judgement of the Investigator) of active non-ocular infection (bacterial, viral, fungal or parasitic) at the Baseline/Day 1 visit or history of severe recurrent bacterial infections.
6. Uncontrolled glaucoma in the study eye defined as intraocular pressure (IOP)>25 mmHG on medication at Screening. Controlled, stable, glaucoma with IOP≤25 mmHG is allowed if mild or moderate (optic nerve abnormalities consistent with glaucoma and glaucomatous visual field abnormalities in one hemifield, and not within 5 degrees of fixation).
7. Presence of diabetic macular edema or history/presence of severe nonproliferative diabetic retinopathy, or proliferative diabetic retinopathy.
8. Vital signs as follows at time of Screening or Baseline/Day 1 visit:
   Body temperature <35.0 or >37.5° C.
   Systolic blood pressure <90 or >180 mm Hg
   Diastolic blood pressure <50 or >110 mm Hg
   Resting pulse rate <50 or >100 bpm. Below 50 bpm is acceptable, if no other clinically significant ECG abnormalities as per investigator decision. For participants with heart rates less than 50 bpm, evidence should be provided that they have no history of a) moderate or severe valvular disease: b) history of coronary artery disease, myocardial infarction, hypertension or diabetes mellitus: c) history of cardiomyopathy, congenital heart defect, open heart surgery, or ongoing arrhythmia: d) family history of sudden death in a first degree relative.
      Note: In the event of an elevated blood pressure measurement, the Investigator may obtain up to two additional readings, so that a total of three consecutive assessments are made with the participant seated quietly for at least 5 minutes before each repeat assessment. At minimum, the last reading must be within range for the participant to qualify.
9. History of clinically significant ECG abnormalities, or any of the following ECG abnormalities at Screening or Baseline/Day 1 visit:
   QTcF >450 msec (males)
   QTcF >460 msec (females)
      History of familial long QT syndrome or known family history of Torsades de Pointes
10. History of stroke or myocardial infarction during the 6-month period prior to Baseline/Day 1, any current clinically significant arrhythmias, or any advanced cardiac or severe pulmonary hypertension.
11. History of kidney failure including end stage renal disease requiring dialysis or renal transplant.
12. History of malignancy of any organ system (other than localized basal cell carcinoma of the skin or in-situ cervical cancer), treated or untreated, within 5 years of Baseline/Day 1, regardless of whether there is evidence of local recurrence or metastases.
13. History of solid organ or bone marrow transplantation.
14. History of recurrent meningitis or history of meningococcal infections despite vaccination.
15. History of immunodeficiency diseases, including a positive HIV test result at Screening.
16. Chronic infection with Hepatitis B (HBV) or Hepatitis C (HCV).
   Participants with positive serology for HBV surface antigen (HBsAg), or if standard local practice, a positive HBV core antigen test, will be excluded.
   Participants with positive serology for HBV core antibody (HBcAb) will be excluded except if the three following criteria are met:
      a) Negative test for HBV DNA.
      b) Prophylactic treatment with lamivudine or entecavir initiated latest on day 1 of treatment and continued until 6 months after last treatment.
      c) Hepatitis B monitoring is implemented: HBsAg and HBV DNA tested every 4 weeks for the first 6 months and every 12 weeks thereafter, until the end of prophylactic treatment.
         Participants with a positive HCV antibody test should have HCV RNA levels measured.
         Participants with positive (detectable) HCV RNA should be excluded.
17. History of hypersensitivity to any of the study treatments or excipients or to drugs of similar chemical classes or clinically relevant sensitivity to fluorescein dye as assessed by the Investigator.
18. Known or suspected hereditary or acquired complement deficiency.
19. History of any porphyria metabolic disorder.
20. Administration of any live vaccination within 4 weeks prior to Baseline/Day 1.
21. Participants previously treated with immunosuppressive or other immunmodulatory agents such as but not limited to cyclophosphamide, rituximab, infliximab, eculizumab, canakinumab, mycophenolate mofetil (MMF) or mycophenolate sodium (MPS), cyclosporine, tacrolimus, sirolimus, everolimus, or systemic corticosteroids exposure (>7.5 mg/d prednisone/prednisolone equivalent) within 90 days (or 180 days for rituximab) prior to first study drug administration.
22. Participants at risk for tuberculosis (TB), specifically participants who:
   Have current clinical, radiographic or laboratory evidence of active or latent TB:
      Evidence of TB infection (active or latent) determined by positive QuantiFERON (QFT) test or positive purified protein derivative (PPD) test (≥5 mm induration) at Screening or within 2 months prior to Screening, according to national guidelines. Participants with a positive or indeterminate QFT test may participate in the study if a full TB work-up (according to local practice/guidelines) completed within 12 weeks prior to randomization, establishes conclusively that the participant has no evidence of active or latent TB. If presence of latent TB infection is established then treatment for TB as per national guidelines must have been initiated or completed prior to Baseline/Day 1. In the absence of national guidelines, the following has been demonstrated: TB has been treated adequately by antibiotics, cure can be demonstrated and risk factors resulting in TB exposure and contracting have been removed.
   Have a history of active TB:
      Within 2 years of Screening, even if it was treated
      Greater than 2 years of Screening, unless there is documentation of adequate treatment according to locally accepted guidelines.
   In the opinion of the Investigator and based upon an appropriate evaluation, have a risk of reactivation of TB that precludes the use of conventional immunosuppression.
23. Use of other investigational drugs within 30 days (e.g. small molecules) or 5 half-lives of Screening or until the expected pharmacodynamic effect has returned to Baseline/Day 1 (e.g. biologics), whichever is longer: or longer if required by local regulations.
24. Previous treatment with gemfibrozil or strong CYP2C8 inhibitors such as clopidogrel within 7 days prior to Baseline/Day 1.
25. Any surgical or medical condition which might significantly alter the absorption, distribution, metabolism, or excretion of drugs, or which may jeopardize the participant in case of participation in the study at Screening or Baseline/Day 1. The investigator should make this determination in consideration of the participant's medical history and/or clinical or laboratory evidence of any of the following:
   A history of invasive infections caused by encapsulated organisms e.g. meningococcus or pneumococcus
   Splenectomy
   Inflammatory bowel disease, peptic ulcers, severe gastrointestinal disorder including rectal bleeding
   Major gastrointestinal tract surgery such as gastrectomy, gastroenterostomy, or bowel resection
   Pancreatic injury or pancreatitis
   Liver disease or liver injury as indicated by any single parameter of ALT (SGPT), AST (SGOT), γ-GT, alkaline phosphatase or serum bilirubin exceeding 2× upper limit of normal (ULN)
   Evidence of urinary obstruction or difficulty in voiding or any urinary tract disorder, that is associated with haematuria
   Severe concurrent co-morbidities, e.g. advanced cardiac disease (NYHA class IV), severe pulmonary arterial hypertension (WHO class IV), or other conditions as judged by the investigator
26. Abnormal results of coagulation panel at Screening suggestive of Disseminated Intravascular Coagulation (DIC).
27. Donation or loss of >400 mL of blood within 8 weeks prior to Baseline/Day 1, or longer if required by local regulation. Plasma donation (>200 mL) within 30 days prior to Baseline/Day 1.
28. Pregnant or nursing (lactating) women, where pregnancy is defined as the state of a female after conception and until the termination of gestation, confirmed by a positive human chorionic gonadotropin (hCG) pregnancy test at Screening or Baseline/Day 1.
29. Women of child-bearing potential, defined as all women physiologically capable of becoming pregnant, unless they are using acceptable methods of contraception during dosing of investigational drug.
   Total abstinence from heterosexual intercourse (when this is in line with the preferred and usual lifestyle of the participant). Periodic abstinence (e.g. calendar, ovulation, symptothermal, post-ovulation methods) and withdrawal are not acceptable methods of contraception.
   Female sterilization (have had surgical bilateral oophorectomy with or without hysterectomy), total hysterectomy or tubal ligation at least six weeks before taking investigational drug. In case of oophorectomy alone, only when the reproductive status of the woman has been confirmed by follow up hormone level assessment.
   Male sterilization (at least 6 months prior to screening). For female participants on the study, the vasectomized male partner should be the sole partner for that participant.
   Barrier methods of contraception: Condom or Occlusive cap (diaphragm or cervical/vault caps). For UK: with spermicidal foam/gel/film/cream/vaginal suppository.
   Use of oral (estrogen and progesterone), injected or implanted hormonal methods of contraception or other forms of hormonal contraception that have comparable efficacy (failure rate <1%), for example hormone vaginal ring or transdermal hormone contraception or placement of an intrauterine device (IUD) or intrauterine system (IUS).

In case of use of oral contraception women should be stable on the same pill for a minimum of 3 months before taking study drug.

If local regulations deviate from the contraception methods listed above and require more extensive measures to prevent pregnancy, local regulations apply and will be described in the informed consent form (ICF).

Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy), total hysterectomy or tubal ligation at least six weeks ago. In the case of oophorectomy alone, only when the reproductive status of the woman has been confirmed by follow up hormone level assessment is she considered not of child-bearing potential.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating age-related macular degeneration (AMD) in an eye of a subject in need thereof, the method comprising orally administering to the subject, iptacopan or a pharmaceutically acceptable salt thereof, at a total dose of about 400 mg daily, wherein the dosing amount refers to the anhydrous free base of iptacopan.

2. The method of claim 1, wherein the AMD is early AMD.

3. The method of claim 1, wherein the AMD is intermediate AMD.

4. The method of claim 1, wherein the iptacopan is administered twice a day.

5. The method of claim 1, wherein iptacopan is administered at a dose of 200 mg twice daily.

6. The method of claim 1, wherein iptacopan is administered for at least one month.

7-9. (canceled)

10. The method of claim 1, wherein the administration of iptacopan reduces vision loss in the eye compared to administration of placebo.

11-14. (canceled)

15. A method of reducing incidence of progression of early or intermediate age-related macular degeneration (AMD) in an eye of a patient in need thereof, the method comprising orally administering to the subject, iptacopan or a pharmaceutically acceptable salt thereof, at a dose of about 400 mg daily, wherein the dosing amount refers to the anhydrous free base of iptacopan.

16. The method of claim 15, wherein the iptacopan is administered twice a day.

17. The method of claim 15, wherein iptacopan is administered at a dose of 200 mg twice daily.

18. The method of claim 15, wherein iptacopan is administered for at least one month.

19-21. (canceled)

22. The method of claim 15, wherein the administration of iptacopan reduces vision loss in the eye compared to administration of placebo.

23-26. (canceled)

27. A method of preventing progression of early or intermediate age-related macular degeneration (AMD) in an eye of a patient in need thereof, the method comprising orally administering to the subject, iptacopan or a pharmaceutically acceptable salt thereof, at a dose of about 400 mg daily, wherein the dosing amount refers to the anhydrous free base of iptacopan.

28. The method of claim 27, wherein the iptacopan is administered twice a day.

29. The method of claim 27, wherein iptacopan is administered at a dose of 200 mg twice daily.

30. The method of claim 27, wherein iptacopan is administered for at least one month.

31-33. (canceled)

34. The method of claim 27, wherein the administration of iptacopan reduces vision loss in the eye compared to administration of placebo.

35-38. (canceled)

39. A method of reducing incidence of atrophic lesions in an eye of a patient in need thereof, the method comprising orally administering to the subject, iptacopan or a pharmaceutically acceptable salt thereof, at a dose of about 400 mg daily, wherein the dosing amount refers to the anhydrous free base of iptacopan.

40. The method of claim 39, wherein the patient suffers from early or intermediate age-related macular degeneration (AMD).

41. The method of claim 39, wherein the iptacopan is administered twice a day.

42. The method of claim 39, wherein iptacopan is administered at a dose of 200 mg twice daily.

43. The method of claim 39, wherein iptacopan is administered for at least one month.

44-46. (canceled)

47. The method of claim 39, wherein the administration of iptacopan reduces vision loss in the eye compared to administration of placebo.

48-52. (canceled)
